(19) United States
(12) Patent Application Publication
(10) Pub. No.: US 2013/0304133 A1
(43) Pub. Date: Nov. 14, 2013

(54) CABLE ANCHOR SYSTEMS AND METHODS

(71) Applicant: Kenneth B. Trauner, San Francisco, CA (US)

(72) Inventor: Kenneth B. Trauner, San Francisco, CA (US)

(21) Appl. No.: 13/893,759

(22) Filed: May 14, 2013

Related U.S. Application Data
(60) Provisional application No. 61/646,675, filed on May 14, 2012.

(51) Int. Cl. A61B 17/82

(52) U.S. Cl. CPC A61B 17/82
USPC 606/301

(57) ABSTRACT

The cable system includes a cable, cable anchors and/or cable crimps. The cable anchors and cable crimps have mounting holes that are used to secure the cable anchors and cable crimps to bones of patients with bone screws. Once the cable anchors and/or cable crimps are attached to the bone, the cable wrapped around the bone and placed through the cable anchors and cable crimps. The cable is tensioned and a locking mechanism is used to lock the cable crimp to the cable. The cable anchors and cable crimps prevent the cable from moving on the bone.

### CROSS REFERENCE TO RELATED APPLICATIONS

This application claims priority to U.S. Provisional Application No. 61/646,675, Systems And Methods For Providing Cable Anchor Systems, filed May 14, 2012 which is hereby incorporated by reference in its entirety.

### BACKGROUND

Cable systems are used to stabilize fractures of bones. Cables can be made of stainless steel pliable cords that are wrapped circumferentially around bone(s). A first end of the cable is free on first end and a second end of the cable is attached to a cable crimp. To use the cable system, the cable crimp which is placed against the bone and the first free end of the cable is wrapped around the bone and coupled to the cable crimp. A separate cable tensioner instrument is then slid over the free end of the cable until the tip of the tensioner is up against the exit hole of the cable crimp. The surgeon uses the tensioner to apply tension to the cable. Once the desired tension is applied, the cable is crimped to the cable crimp. After the cable has been crimped, the cable tensioner is removed from the cable and the cable is cut flush with the cable crimp. The cable and the cable crimp can remain in the body permanently.

The problem with the current existing cable crimp part is that the size of the part is too tall and prominent and leads to symptomology for patients, such as pain and palpable tenderness over the cable crimp. In addition, the cable crimp system tends to loosen, migrate and shift after it has been placed either intraoperatively or post-surgery.

Current cable systems face other limitations. The cable requires secure placement on a relatively uniform diameter surface to maintain its position. If the cable is tensioned or secured on a tapered cross section of bone, the cable frequently displaces towards the direction of lesser cross section. With displacement, tension is lost in the cable and as is the mechanical stability that the cable intended to provide. Displacement of cables and loss of fixation of the bone constructs is a frequent complication with use of current cable systems. This issue is particularly relevant in hip surgery. In hip surgery, the trochanter is frequently cut or osteotomized for access to implants in the inner canal and the trochanter is also frequently a site of fracture. Because implants are usually retained inside the bone, use of plate and screw constructs that bridge both cortices of the shaft are not possible. Cables are frequently used, however, the proximal femoral metaphyseal region of the bone has a tapered morphology that tends to allow for displacement of the cables.

What is needed is an improved cable crimp that does not cause pain or tenderness and prevents the cable from loosening or migrating.

### SUMMARY OF THE INVENTION

The cable system includes a cable, cable anchors and/or cable crimps. The cable can be wrapped around a bone and held in a specific position in tension to stabilize a bone fracture. Cable anchors and cable crimps can be used to hold the cable in tension at specific areas of the bones.

When the cable anchor is need, the surgeon determines the desired location(s) of the cable anchor(s). The surgeon can select the appropriate bone screw and installation location. The cable anchor can then be attached to the bone by screwing the bone screw into the bone. The Cable Anchor is screwed into the bone through the cortex of the bone into the cancellous region. However if a tack type of screw is selected, then the bone screw may only penetrate the cortex region of the bone.

A cable stop may be mounted on one end of the cable that prevents the cable from sliding through a cable anchor. However, in other embodiments, the cable anchor may not come with the pre-run cable. The cable can run free through pass-through cable anchors and cable crimps can be used to prevent the cable from sliding and maintain cable tension in the cable system. A free end of the cable can be wrapped around bone and through the holes in the cable anchor(s). The free end of the cable can then placed through the cable anchor crimp. The cable is tensioned, crimped to hold the cable in the desired tension. After the cable is tensioned and secured to the cable crimp, the cable can be cut and the excess cable can be removed.

The inventive cable crimp and cable anchor provide various benefits over prior art cable systems. In order to prevent cable movement, the inventive system uses bone screws to fixate the cable crimp and the cable anchor into a bone of the patient. The bone screws prevent shifting and migration of the cables and prevents the cable from losing tension. The cable tension can be critical to the success of the surgery. If the cable loses tension, the cable can shift and the cable may no longer properly stabilized the bone fracture. The inventive cable system provides improved fracture stabilization, compression, reduction and fixation which will not lose tension.

The screw fixation can insure that the cable remains in the proper position. The screws used to secure the cable crimp and the cable anchor can be chosen from multiple screw types, lengths, pitch and heads. The ability to select different screws allows for surgeon discretion about what type of pull out strength desired for the individual cable system being placed.

Another advantage of the inventive cable anchors and cable crimps is the lower profile body, less prominent, and a “button” style design. This low profile design can be positioned flatter against the bone and the structures can have rounded, softer edges. The placement location(s) of cable anchor and cable crimp in the body tends to be in areas that have less fat and soft tissue. The lower profile, softer part design applies less pressure to surrounding soft tissue, leads to less reaction and inflammation and results in improved patient satisfaction.

The inventive cable system that uses cable anchors together with the cable anchor crimps allows can provide more options to stabilize more types of bone fractures over greater distances and more complex bone geometries. The inventive cable system can replace the need for bulky, symptomatic bone plates and screws. The cable anchor can have a smooth “button” design that allows for easier surgeon placement in areas of bone. The inventive cable anchor can be passed through tight spaces and pressed into the bone using the compression anchor tacks which can extend from the lower surfaces of the inventive cable anchors and cable crimps and can further stabilize the cable anchors and cable crimps on the bone.

The inventive cable system can be used for various types of bone stabilization. A typical location for use of the inventive cable system can include stabilization of trochanteric fractures of the femur or use as a circular construct around fractured or non-intact long bones. To create the circular construct, a cable is wrapped circumferentially around the long bone and both ends of the cable are passed through cable locking mechanisms. A cable locking mechanism can be a cable stop which can be a structure attached to the end of the cable. The opposite end of the cable can be secured to a cable crimp locking mechanism. With the cable situated in the desired location, tension is applied to the cable. The locking mechanism of the cable crimp can be engaged to secure the tension in the cable. The cable tension provides circumferential compression to the underlying bone or bone grafts.

Maintaining the position of the cable in a specific location while tensioning the cable is also frequently a challenge for the surgeon. As the cable is tensioned, it has a tendency to migrate to its most stable resting position. Unfortunately, the location of migration is not always the best location to capture bone fragments, bone graft or plate constructs.

This invention describes an array of cable anchor designs and constructs for stabilization of the cables in multiple settings. The multiple designs allow the surgeon to vary choice of implant depending on the surgical access to the site, the mechanical properties and quality of the host bone, the resistance to pull out, the need to adjust the anchor once placed. This invention describes a cable anchor system that allow the surgeon to secure the surgical cables to the bone at any location with low profile cable anchors.

The present invention includes a variety of constructs for specific locations such as the greater trochanter where use of multiple cables and anchors used in conjunction provides mechanical stability and greater resistant to rotational displacement of the bone over existing cable approaches. This invention describes use of anchor mechanical construct combined with a cannulated cable stabilization component that is applied to the cable and can allow the cable to be attached to the bone at varied locations along its length. The use of these cable systems also provides a lower profile trochanteric stabilization system compared to existing cable plate systems. This improved cable stabilization reduces the motion and displacement of cables which frequently leads to fretting of the cables. The frayed cables causes irritation of surrounding muscle and soft tissue and frequently leads to additional surgery for cable removal.

In another embodiment the anchor is attached to a site other than the crimping mechanism. The cable is passed circumferentially around the bone and passed through the anchor bolt or anchor loop. The cable is tensioned and locked at a separate location from the anchor with a crimp or screw crimp construct. The anchor, or multiple anchors prevent displacement of the cable.

Anchors can have multiple configurations to prevent backing out of the bone and can utilize bone screws and bone tacks. The inventive cable anchors and cable crimps can also be modular assemblies to allow the surgeon greater flexibility in the design of the cable systems.

In an embodiment, the cable is be secured at one end with a ball tipped cable stop which prevents the cable from passing through a smaller diameter or smaller width opening. The cable stop can provide a “drop in” configuration at one end so that the opposite end of the cable can be tensioned alone. The remaining cable is then passed can be passed longitudinally along the bone to a second site where the cable is secured to a cable crimp after tensioning by a cable anchor crimping device. Additional anchors can be placed anywhere along the bone to secure the position of any cable.

In another construct two cables can be rigidly coupled together and passed from the lateral side of the femur under the lesser trochanter on the medial side. The cables are placed obliquely and frequently displaced. The two cables are connected at near midpoint and are passed together around the medial side the separated longitudinally on the lateral side. The use of the medial connection of the two cables secures each from displacement. Used in conjunction with cable anchors and with a low profile longitudinal construct provides a stable triangular construct that has improved resistance to displacement, lowered risk of trochanteric bursitis and greater resistance to mechanical rotation about the long axis of the femur. Greater stability to rotation minimizes the risk of trochanteric nonunion or failure at the fracture to due to excessive motion of the fracture site.

For any of the positioning devices, crimping of the cable at the second location will change the tension profile of the cable and prevent translation within the positioner and minimize risk of fretting. The cable anchor crimp part and/or the cable anchor part can have cable holes that are not radiused. In other embodiments, the cable holes can be radiused to prevent fretting. The radius refers to the edges of the hole through which the cable passes. The radius can be important if there is any sort of repeated translation to prevent fretting. If the cable is secured within the cable anchor or cable crimp, the radius is less critical since there may not be any longitudinal motion at the interface.

In some embodiments, the modular cable anchor can have a bone screw portion and a cable anchor or cable crimp portion. Since this can be a modular system, the cable anchor or crimp can be manufactured as a separate part from the bone screw. The anchoring of the assembly of the modular structures can be done ex vivo or in situ by the surgeon. All of these components can be made of certified medical grade 316 stainless steel, any other suitable grade stainless steel, titanium, titanium alloy or any other suitable material or composition of materials can be used for the cables, cable anchors and cable crimps.

Currently cable systems are used almost exclusively for circular constructs with the cable passed around the bone circumferentially and secured to itself. The inventive cable system utilizes bone coupling devices to keep the cables in place on the bone. By coupling the cable anchors and cable crimps to the bone, cable systems to be designed with the cables extending across portions of the bone at any desired positions and angles. Because the cables anchors and cable crimps are secured to the bone the cable system can be arranged in any configuration across any portion of the bones without having the cable come loose. Thus, the cable does not have to be wrapped around the entire circumference of the bone or be connected to itself to provide the required compression to the bone.

### BRIEF DESCRIPTION OF THE DRAWINGS

FIG. 1 illustrates a top view of an embodiment of a cable anchor crimp;

FIGS. 2 and 3 illustrate side views of an embodiment of a cable anchor crimp;

FIG. 4 illustrates a bottom view of an embodiment of a cable anchor crimp;

FIG. 5 illustrates a cross section side view of an embodiment of a cable anchor crimp on a bone;

FIG. 6 illustrates a cross section side view of an embodiment of a cable and a cable anchor crimp on a bone;

FIG. 7 illustrates a cross section top view of an embodiment of a cable and a cable anchor crimp;

FIGS. 8 and 9 illustrates cross section side view of an embodiment of a cable and a cable anchor crimp;

FIG. 10 illustrates a top view of an embodiment of a cable anchor;

FIG. 11 illustrates a side view of an embodiment of a cable anchor;

FIGS. 12 and 13 illustrate side views of embodiments of cable anchors on bones;

FIGS. 14-19 illustrate perspective views of alternative embodiments of cable anchors;

FIG. 20 illustrates a side view of an embodiment of a cable anchor;

FIG. 21 illustrates a perspective view of an embodiment of a cable anchor;

FIG. 22 illustrates a cross section top view of an embodiment of a cable anchor;

FIG. 23 illustrates a side view of an embodiment of a cable anchor;

FIG. 24 illustrates a perspective view of an embodiment of a cable anchor;

FIG. 25 illustrates a top view of an embodiment of a cable anchor;

FIGS. 26 and 27 illustrate perspective views of embodiments of modular cable anchors;

FIG. 28 illustrates a side view of an embodiment of a bone screw;

FIG. 29 illustrates a cross section side view of an embodiment of a bone screw;

FIGS. 30A and 30B illustrate top views of different embodiments of a bone screw;

FIG. 31 illustrates a side view of cable anchor insert;

FIG. 32 illustrates a bottom view of a cable anchor insert;

FIGS. 33 and 34 illustrate side views of a bone screw and a cable anchor installation;

FIGS. 35-42 illustrate top and side views of different embodiment of modular cable anchors and cable crimp inserts;

FIGS. 43-50 illustrate top and side views of different embodiment of low profile cable anchors and cable crimps;

FIG. 51 illustrates an embodiment of a cable system on a bone;

FIG. 52 illustrates two cables rigidly coupled together with a connector;

FIG. 53 illustrates an embodiment of a trochenteric cable fixation system;

FIG. 54 illustrates an embodiment of a cable system for a structural allograft;

FIG. 55 illustrates a cross section view of the cable system for a structural allograft; and

FIGS. 56 and 57 illustrates side views of a strut graft held to a bone with an embodiment of the cable system.

### DETAILED DESCRIPTION

The inventive cable anchor system can include: a cable, a cable crimp and the cable anchor. The cable can be made of many strands of metal wire that are laid or twisted into a helix manner. Because the cable consists of many thin diameter pieces of wire, the cable is flexible and can bend in smaller radius than a solid metal rod.

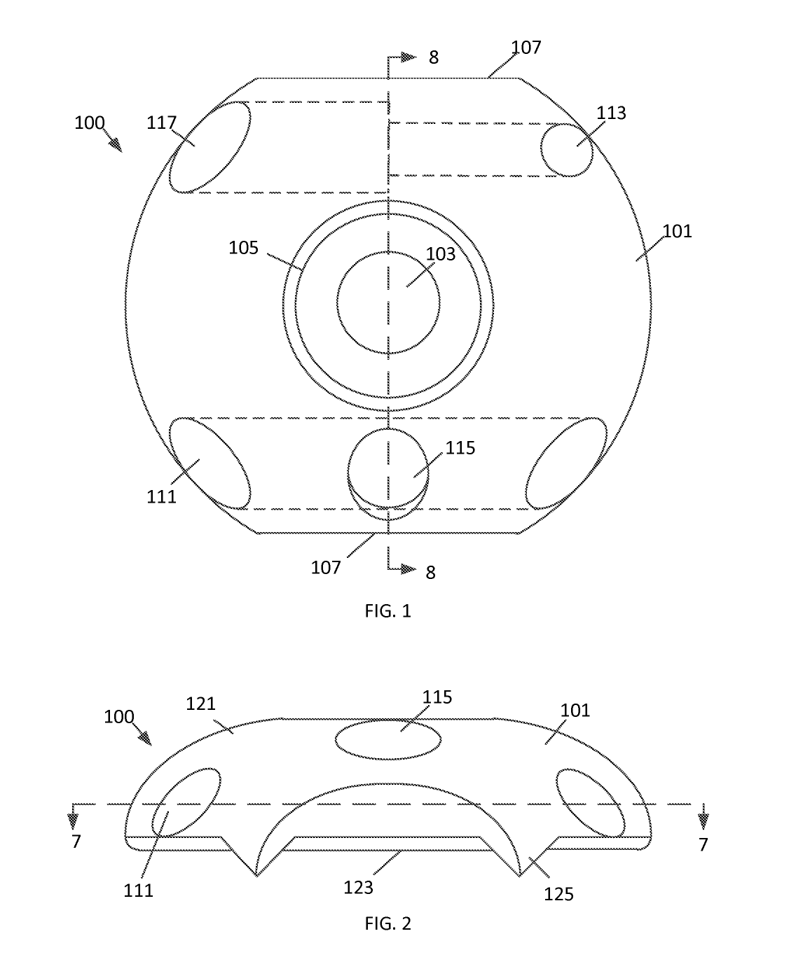

With reference to FIGS. 1-4 different views of an embodiment of a cable crimp 101 are illustrated. FIG. 1 illustrates a top view of the cable crimp 101. In this embodiment, the cable crimp 100 has a circular body 101 having a center hole that extends through the center of the body 101. The upper portion of the center hole 105 can have a wider diameter than the lower portion of the center hole 103. The body 101 can have flat sections 107 on opposite sides of the body 101 that can be used to grip and rotate the body 101. The body 101 can also have a first cable hole 111 and a second cable hole 113 that extend horizontally through the body 101. The first cable hole 111 can have a uniform diameter and may be substantially perpendicular to the center hole 103. A locking hole 115 can intersect the first cable hole 111. The second cable hole 113 can be aligned with a counter bore 117 which has a larger diameter than the second cable hole 113. Both the locking hole 115 and the counter bore 117 can be used to hold the cable to the cable crimp 101.

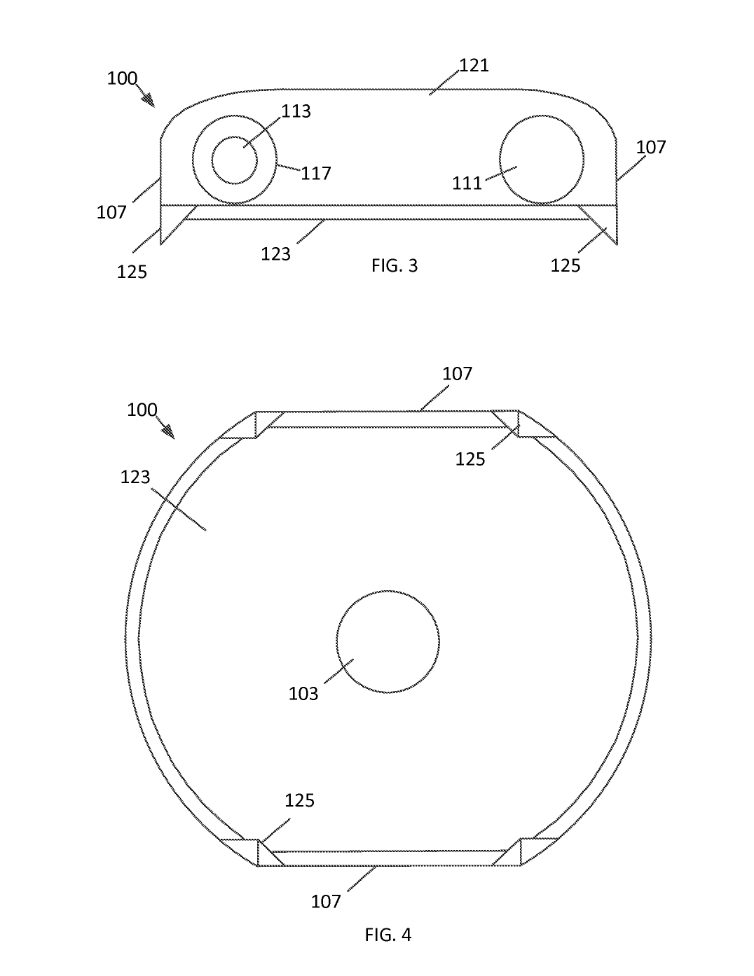

FIGS. 2 and 3 illustrates side views of the cable crimp 100. In this embodiment, the upper surface 121 can be a convex surface and lower surface 123 of the cable crimp body 101 can be a flat or concave surface. The edge of the body 101 where the upper surface 121 and the lower surface 123 intersect, can have a chamfer or a radius so that this area does not have sharp edges. A plurality of compression anchor tacks 125 can extend from the lower surface 123 of the body 101. The compression anchor tacks 125 can be pressed into the bone of the patient when the cable crimp 100 during the cable system surgery. FIG. 3 illustrates the alignment of the second cable hole 113 with the larger diameter counter bore 117. FIG. 4 illustrates a bottom view of the cable crimp 100. The compression anchor tacks 125 can be positioned around the perimeter of the body 101.

FIG. 5 illustrates a cross sectional side view of the cable crimp 100 installed on a bone 200. The bone can be pre-drilled for the bone screw 130. The cable crimp 100 can then be placed over the bone 200 and the bone screw can be placed with a bone screw 130 placed through the lower portion of the center hole 103. The head of the bone screw 130 can fit within the upper portion of the center hole 105 can have a wider diameter than the lower portion of the center hole 103. The bone screw 130 can be threaded and screwed into the bone. The force of the bone screw 130 can drive the compression anchor tacks 125 into the bone 200. The bone screw 130 can hold the cable crimp 100 to the bone 200 and the compression anchor tacks 125 can prevent the cable crimp 100 from moving or rotating.

In different embodiments, a drill hole is created to a know depth in one cortex of the femur or long bone using a step drill with a know penetration into the bone. The anchor is seated in the bone. This invention describes several mechanisms for anchoring to the bone-drill and use of self tapping anchors with thread, drill and tap with then placement of a threaded anchor, use of drill with an anchor that is press fit into the bone.

FIG. 6 illustrates a side view of the cable crimp 100 installed on a bone 200. A cable 150 has been attached to the second cable hole 113, wrapped around the circumference of the bone 200, tensioned and passed through the first cable hole 111. In this embodiment, a threaded lock bolt 116 is screwed into the threaded locking hole and the end of the lock bolt 116 is pressed against the cable 150. The cable 150 is deformed and the compression of the cable against the inner diameter of the first cable hole 111 and the end of the lock bolt 116 holds the cable 150 in a static position within the first cable hole 111.

The cables used with the inventive cable system can include multiple strands of metallic material woven to provide high levels of tensile strength. The cables as a composite of multiple strands is resistant to fatigue and tolerates repetitive loads for prolonged duration while maintaining its tensile properties. Wires such as 18 gauge luque wires are frequently used for cable constructs around bone. To create the wire construct, a large wire such as 18 gauge wire is passed around the femur. In an embodiment, the cable tension is provided by twisting the wire while pulling on the wires with a pair of specialized pliers. In other embodiments, any other suitable cable material that can be assembled into a high strength cable used with the inventive system including advanced composite materials such as Kevlar fibers, carbon fibers, etc.

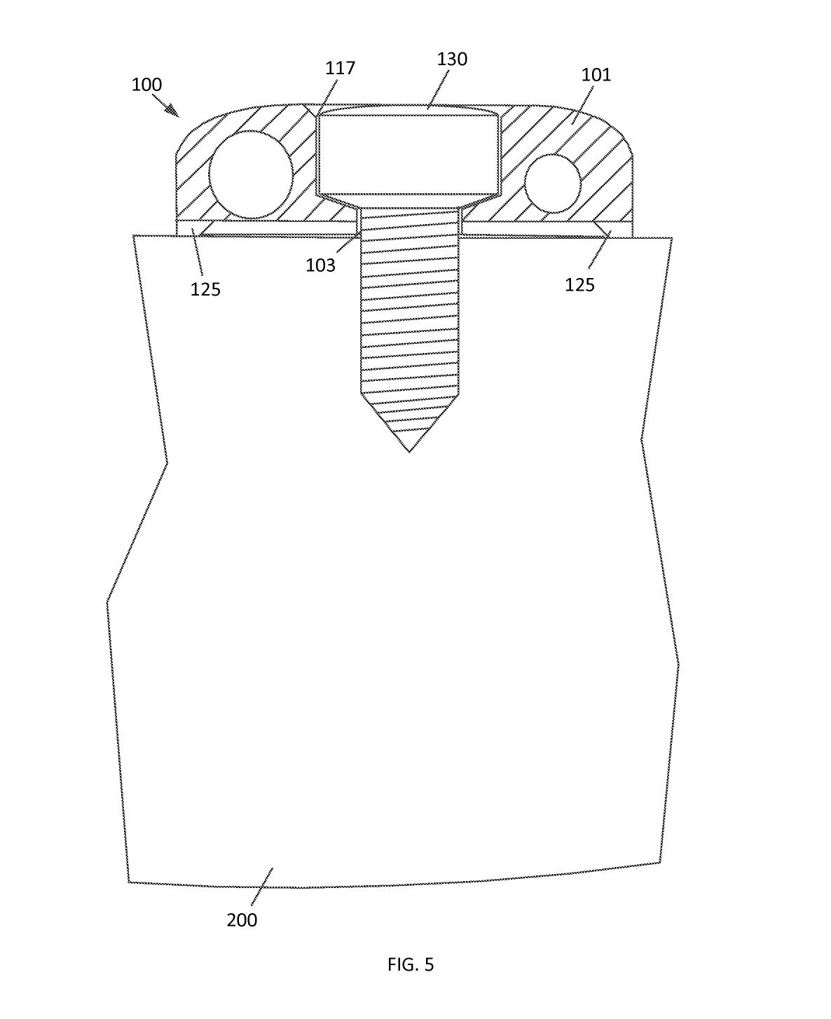

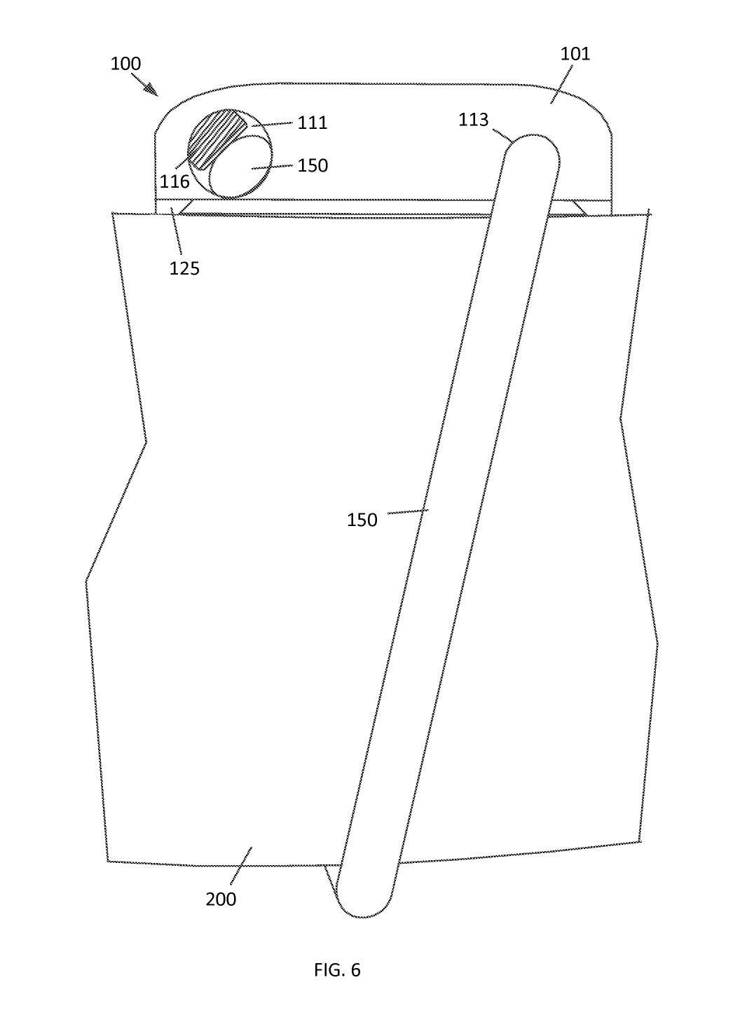

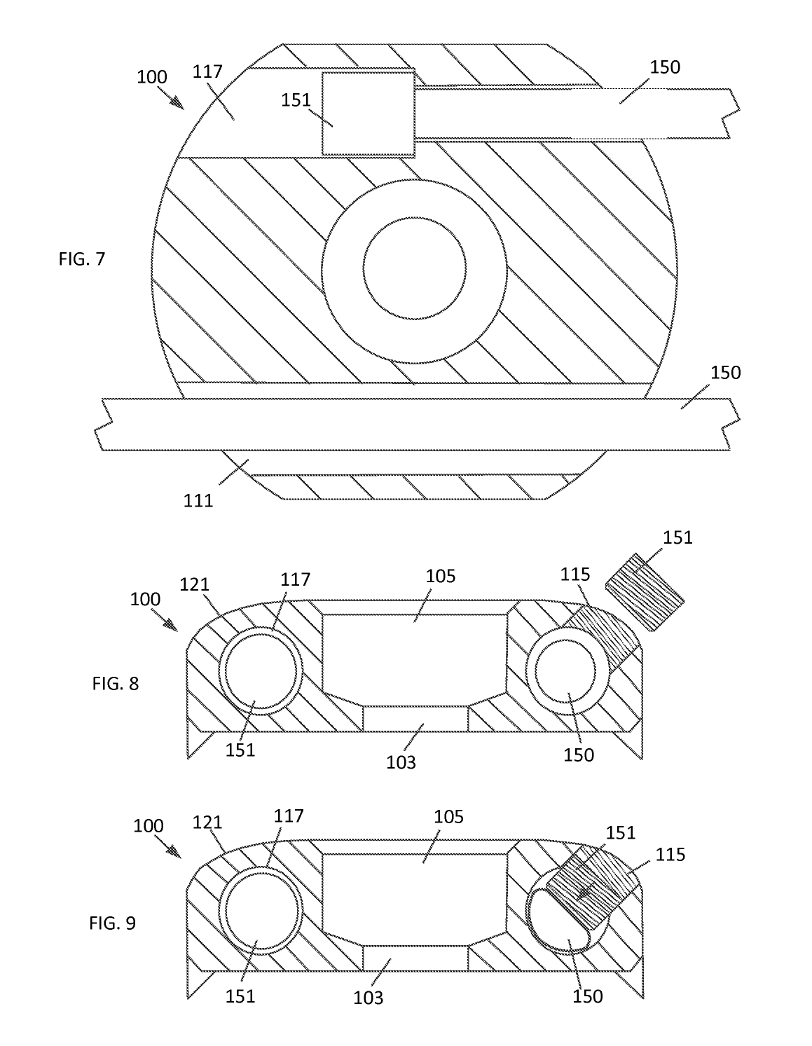

FIG. 7 illustrates a cross section top view of the cable crimp 100 and a cable 150. In this embodiment, a cable stop 151 is attached to one end of the cable 150. The cable stop 151 can be cylindrical in shape. The width of the cable stop 151 can be a wider diameter than the second cable hole 113 but smaller than the counter bore 117. Thus, when the cable 150 is pulled through the second cable hole 113, the stop 151 will hold the cable 150 in tension. The opposite end of the cable 150 can be wrapped around the bone and threaded through the first cable hole 111 and pulled in tension with a tensioner. FIGS. 8 and 9 illustrate a side view of the cable crimp 100 with the cable 150 placed through the first cable hole 111. The cable 150 can be tensions and a locking bolt 151 can be screwed into the threaded locking hole 115. The top end of the locking bolt can have a recessed driving surface such as slots for a screw driver, a hexagonal recess for an hex wrench, a torx recess for a torx wrench, or another suitable driving surface for rotating the locking bolt 151. With reference to FIG. 9, the locking bolt 151 can be tightened and compressed against the cable 150 which causes the cross section of the cable 150 to be crushed and deformed. This compression crimps the cable 150 to the cable crimp 100 and keeps the cable 150 in tension.

When the cable anchor crimp is used, the surgeon determines the desired location(s) of the cable system. The cable anchor crimp can be provided with a cable pre-run through one of the two holes of the body with the cable end is locked into one hole of the part. The surgeon first secures the cable anchor crimp into the bone with a bone screw which penetrates through the outer cortex of the bone into the cancellous region of the bone. The surgeon then wraps the cable around the bone and runs the cable through the open hole where the locking bolt screw resides. At this point the surgeon applies the cable tensioner over the loose cable and adjacent to the exit hole of the cable anchor crimp. The surgeon can lock the tensioner and apply the desired amount of tension. After the proper cable tension is applied, the locking bolt can be screwed into the body of the cable anchor crimp until the cable is compressed and rigidly held within the cable anchor crimp. The tensioner is then removed and the cable is then cut flush at the exit hole of the part.

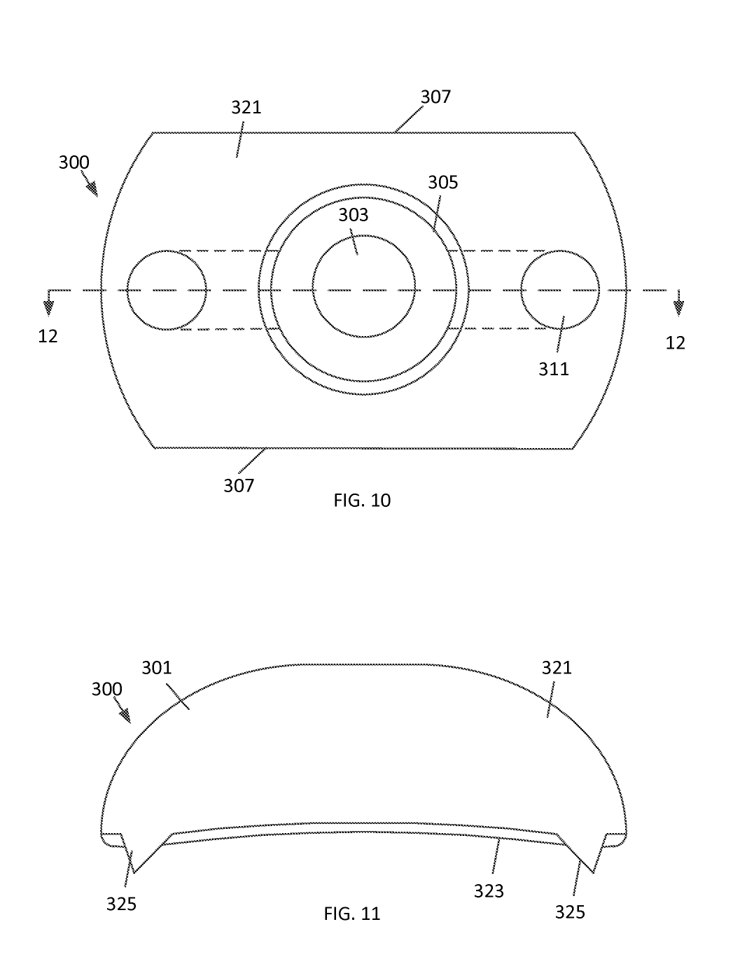

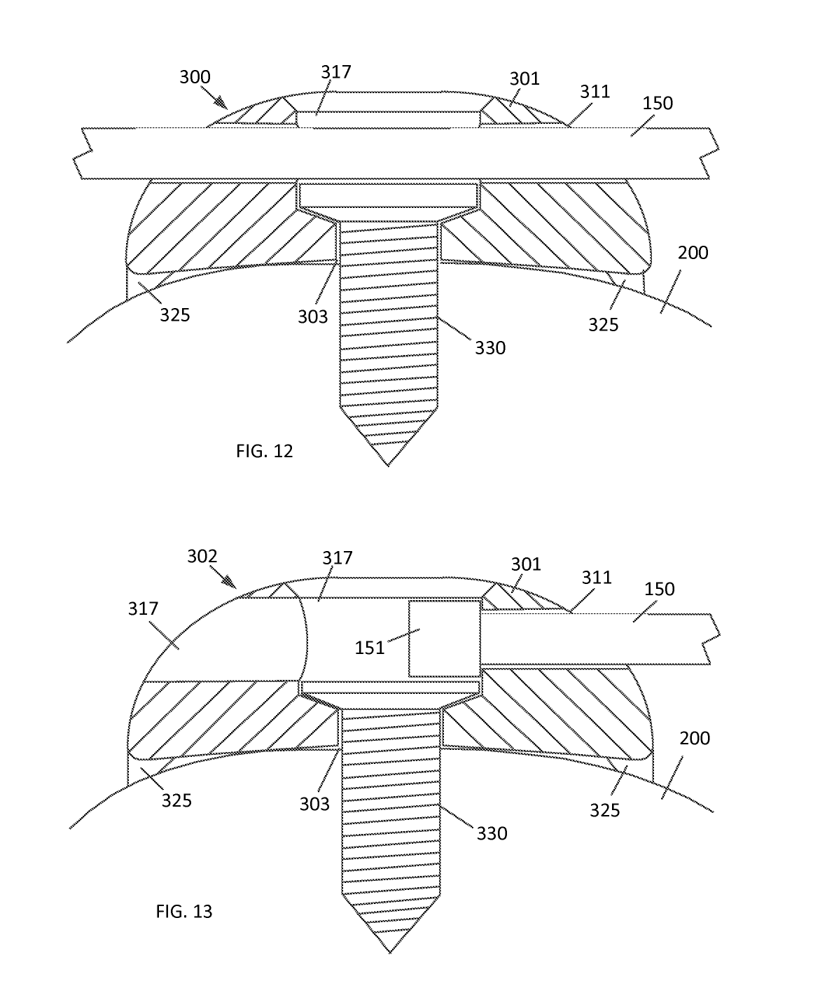

FIGS. 10-12 illustrate embodiments of a cable anchor 300 which can hold the cable 150 to the bone 200 of the patient. The cable anchor 300 can provide an anchor point that prevents the cable 150 from moving over the surface of the bone. The cable anchor 300 can be used in combination with the cable 150 and the cable crimp 100 to hold the cable 150 in the desired tension and position around a bone 200 of the patient. FIG. 10 illustrates a top view of an embodiment of a cable anchor 300. The cable anchor 300 can be similar to the cable crimp. In the illustrated embodiment, the cable anchor 300 has a circular body 301 with flats 307 on opposite sides of the body 301. The center of the cable anchor 300 can have a hole having an upper section 305 with a larger diameter and a lower section 303 with a smaller diameter. A cable hole 311 can extend through the body 301 and can intersect the upper section 305 of the center hole. FIG. 11 illustrates a side view of the cable anchor 300. The upper surface 321 can have a convex surface and the lower surface 323 can have a concave or flat surface. Anchor compression tacks 325 can extend down from the lower surface 323. FIG. 12 illustrates a cross section side view of the cable anchor 300 attached to a surface of a bone 200 with a bone screw 330 and a cable 150 placed through the cable hole 311.

With reference to FIG. 13, a cross section of an alternative embodiment of the cable anchor 302 is illustrated. In this embodiment, the body 303 can have a first cable hole 311 having a first diameter and a second cable hole 317 having a second diameter that is larger than the first diameter of the first cable hole 311. Both the first cable hole 311 and the second cable hole 317 can intersect the upper portion of the center hole 317. The second diameter of the second cable hole 317 can be larger than the diameter of a cable stop 151 attached to the end of the cable 150. After the cable anchor 302 is attached to the bone 200 with the bone screw 330, the cable 150 can be threaded and pulled through the second cable hole 317 and the first cable hole 311. The cable stop 151 can pass through the second cable hole 317 but can be stopped at the first cable hole 311. Since the tension is aligned with the center hole, the cable tension will not excerpt a rotational force on the cable anchor 302.

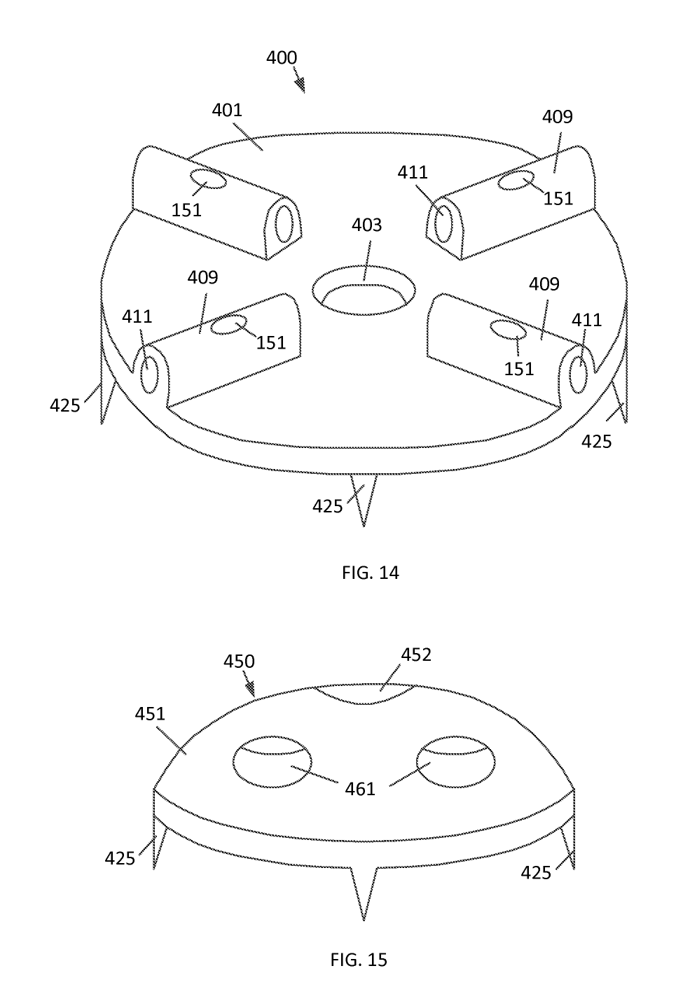

In other embodiments, the cable anchors and cable crimps can have various other configurations. With reference to FIG. 14, a cable anchor 400 is illustrated having a disk shaped body 401 with a plurality of cable holding structures on the upper surface arranged in a radial pattern from the center of the body. The cable anchor 400 can include a center hole 403 for securing the cable anchor 400 to a bone with a bone screw. Compression anchor tacks 425 mounted around the lower circumference of the body 401 can be pushed into the surface of the bone to hold the cable anchor 400 in place. A cable(s) can be threaded through holes 411 in the aligned cable holding structures 409 across the body 401 and the cable anchor 400 can hold the cable in the desired position on the bone. In an embodiment, the cable holding structure 409 can include locking bolts 151 that are tightened against the cables within the cable holding structures 409 to maintain the cable tension.

For some locations, surgical exposure is insufficient to allow for drilling of the bone. Use of a threaded cable anchor is not possible. In locations such as the medial border of the proximal femur, curved cable passers are used to pass the cable beneath the critical structures such as arteries, veins, nerves etc. proper placement of the cables is confirmed by manually by feel. To stabilize in these locations, one embodiment is use of a compression cable anchor tack. The cable passes through a fenestration in the tack, the tack secures to a clamp that is specifically configured to reach around the bone such as a verbrugge clamp. The tack is secured to the clamp for placement. Once properly positioned, the clamp is compressed driving the sharp leading edge into the bone to prevent lateral displacement. With reference to FIG. 15, a cable anchor 450 is illustrated having circular body 451 and a convex dome upper surface. A plurality of cable holes that run through the body 451. The cable anchor can have a plurality of compression anchor tacks 425 around the lower circumference of the cable anchor body 451 and may not include center hole for a bone screw. The cable anchor 450 can be attached to the bone with the verbrugge clamp.

The cable anchor 450 is configured to be low profile, to resist lateral displacement with sharp anchor tacks 425. In other embodiments, the cable anchor 450 may include a central tack surrounded by lower profile anchor tacks 425 to prevent lateral translation of the cable anchor 450. The anchor tacks 425 are configured on one embodiment not to extend beyond the outer diameter of the head of the device in order to minimize risk of glove penetration by the surgeon. The verbrugge clamp can be secured to a coupling recess 452 and the verbrugge clamp can include a release mechanism to prevent displacement of the cable anchor 450 until the anchor tacks 425 are secured to the bone.

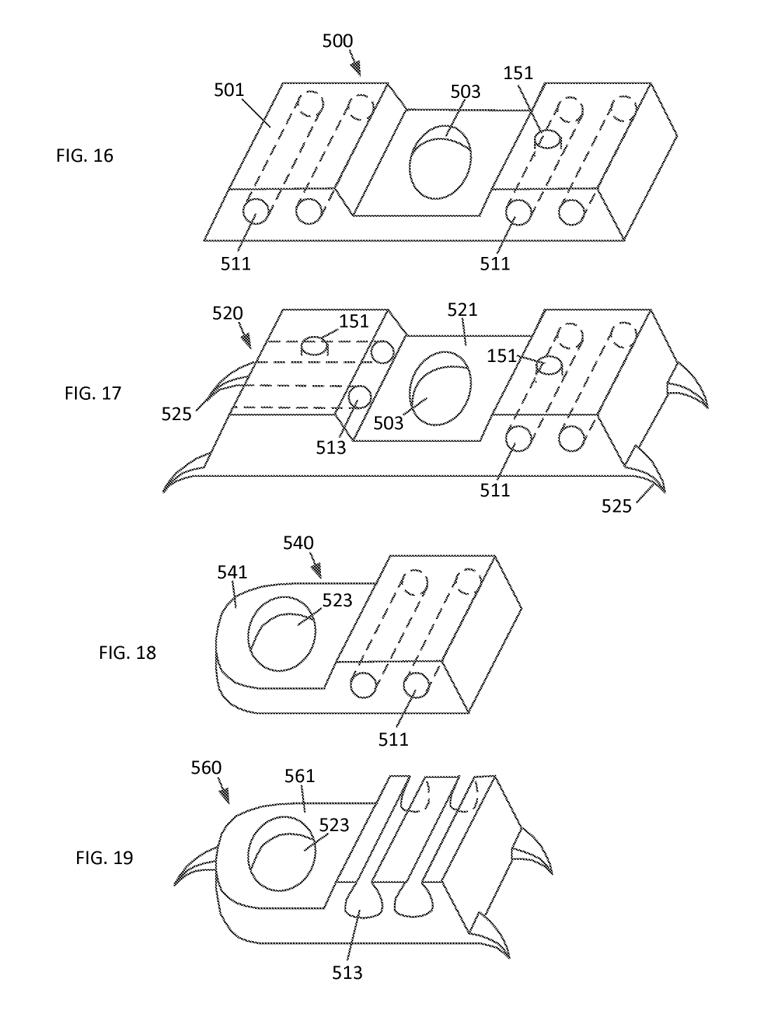

With reference to FIGS. 16-19 other embodiments of the inventive cable anchor are illustrated. FIG. 16 illustrates an embodiment of the cable anchor 500 having a rectangular body 501 and a center hole 503 for securing the cable anchor 500 to a bone. The center hole 503 can be on a central thinner section of the body 501. A plurality of cable through holes 511 extend across the width of the cable anchor body 501 in parallel the thicker end sections of the body 501. FIG. 17 illustrates another embodiments of the cable anchor 520 having a body 521 with some cable holes 511 extending across the width and some cable holes 513 extending in alignment with the length. Compression anchor tacks 525 can extend downward from the sides of the body 521 that can pierce the surface of the bone. In different embodiments, the cable holes 511, 513 can have locking bolts 151 that can be tightened to lock the cable to the cable anchors 500, 520.

FIG. 18 illustrates a cable anchor 540 having a body 541 with a mounting hole 523 on one side and a plurality of cable holes 511 on the opposite side of the body 541. FIG. 19 illustrates a cable anchor 560 having a body 561 with a plurality of cable slots 513 that extend across the width of the cable anchor body 561. Compression anchor tacks 525 that can extend downward from the sides of the body 561 that can pierce the surface of the bone when the cable anchor 560 is mounted on the bone. The cable can be placed in the cable slots 513 and prevent the cable from sliding across the surface of the bone.

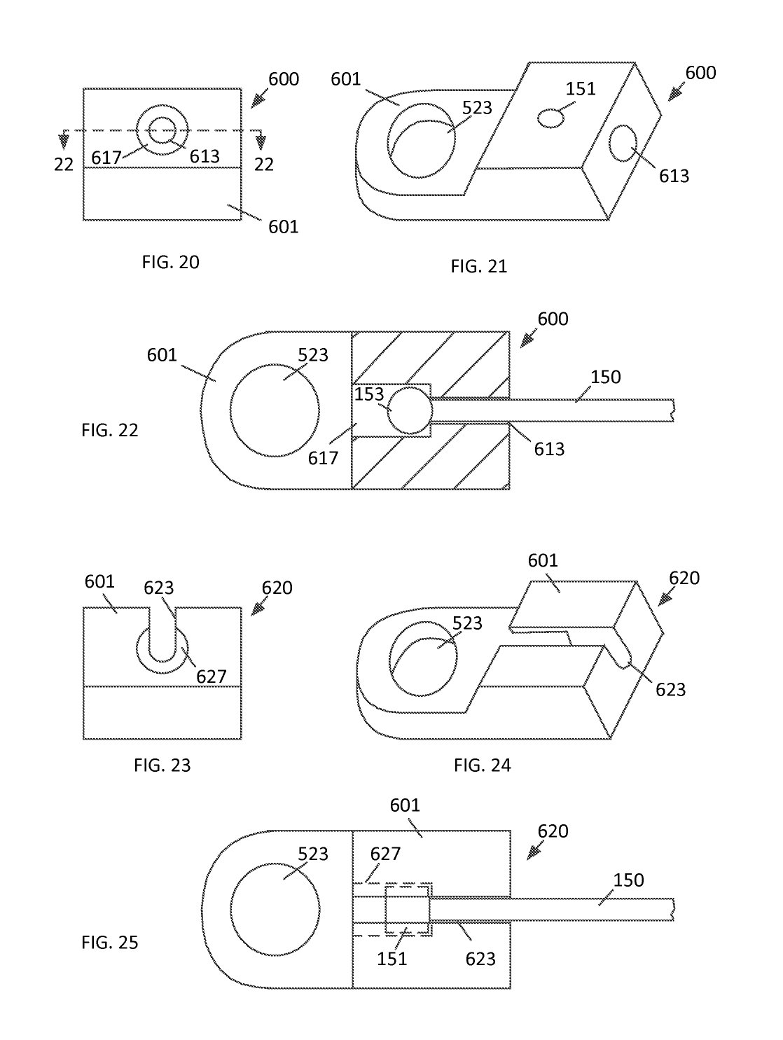

With reference to FIGS. 20-25, embodiments of cable crimps are illustrated. FIG. 20 is a side view of an embodiment of a cable crimp 600 having a cable hole 613 and a counter bore section 617 that are aligned with the length of the cable anchor 600. FIG. 21 illustrates a perspective view of the cable crimp 600 showing the mounting hole 523 for securing the cable crimp 600 to a bone with a bone screw. In an embodiment, the cable crimp can also include a locking bolt 151 that can be used to rigidly secure a cable to the cable crimp 600. This configuration can allow the cable crimp 600 to be rigidly secured to cables that do not have cable stops. FIG. 22 illustrates a cross section top view of the cable crimp 600 with a cable 150 placed through the cable hole 613. A ball stop 153 having a spherical surface is rigidly attached to the end of the cable 150. The ball stop 153 can have a smaller diameter than the diameter of the counter bore 617. Thus, when the cable 150 is tensioned the ball stop 153 can enter the counter bore 617 but can be stopped at the edge of the cable hole 613.

In some embodiments, the cable crimp can use a cable slot rather than a cable hole. With reference to FIGS. 23-25 an embodiment of a cable crimp 620 having a body 621 having mounting hole 523 on one side and thicker portion of the body with a mounting cable slot 623 on an opposite side. The slot can be aligned with the length of the cable crimp 620 and the mounting hole 523. The body may also include a counter bore 627. The cable 150 can be placed into the slot and a cylindrical cable stop 151 on the end of the cable 150 can fit within the counter bore 627. When the cable 150 is tensioned, the stop 151 can be pulled against the intersection of the cable slot 623 and the counter bore 627.

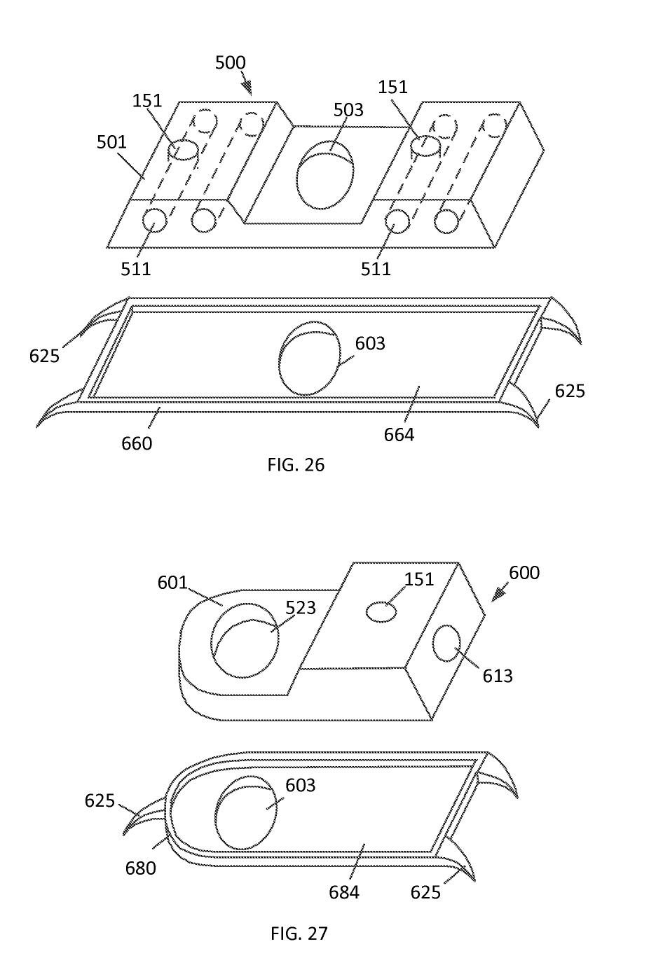

With reference to FIGS. 26 and 27, the inventive cable anchors and cable clamps can be modular systems having multiple components. With reference to FIG. 26, the cable anchor 500 can be combined with an anchor tack plate 660. In this embodiment, the cable anchor 500 can be placed over the anchor tack plate 660. In this embodiment, the anchor tack plate 660 can have a recessed area 664 on the upper surface that corresponds to the lower surface and perimeter of the cable anchor 500. When the cable anchor 500 is within the recessed area 664, the cable anchor 500 cannot rotate relative to the anchor tack plate 660. In other embodiments, other mechanisms can be used to prevent rotational movement between the cable anchor 500 and the anchor tack plate 660. A bone screw can be placed through the center hole 503 in the cable anchor 500 and the center hole 663 in the anchor tack plate 660. When the bone screw is screwed into the bone, the head of the screw can compress the cable anchor 500 and the anchor tack plate 660 against the bone. The anchor tack plate 660 can have a plurality of compression anchor tacks 625 that extend downward from the sides or bottom of the anchor tack plate 660 that can penetrate the surface of the bone and prevent movement and rotation of the cable anchor 500.

With reference to FIG. 27, a cable anchor 600 and an anchor tack plate 680 are illustrated. The anchor tack plate 680 can include compression anchor tacks 625 that extend downward from the sides or bottom of the anchor tack plate 680. A recessed area 684 on the upper portion of the anchor tack plate 680 can surround the lower perimeter of the cable anchor 600 and prevent it from rotating when the cable anchor 600 and the anchor tack plate 680 are secured to a bone with a bone screw.

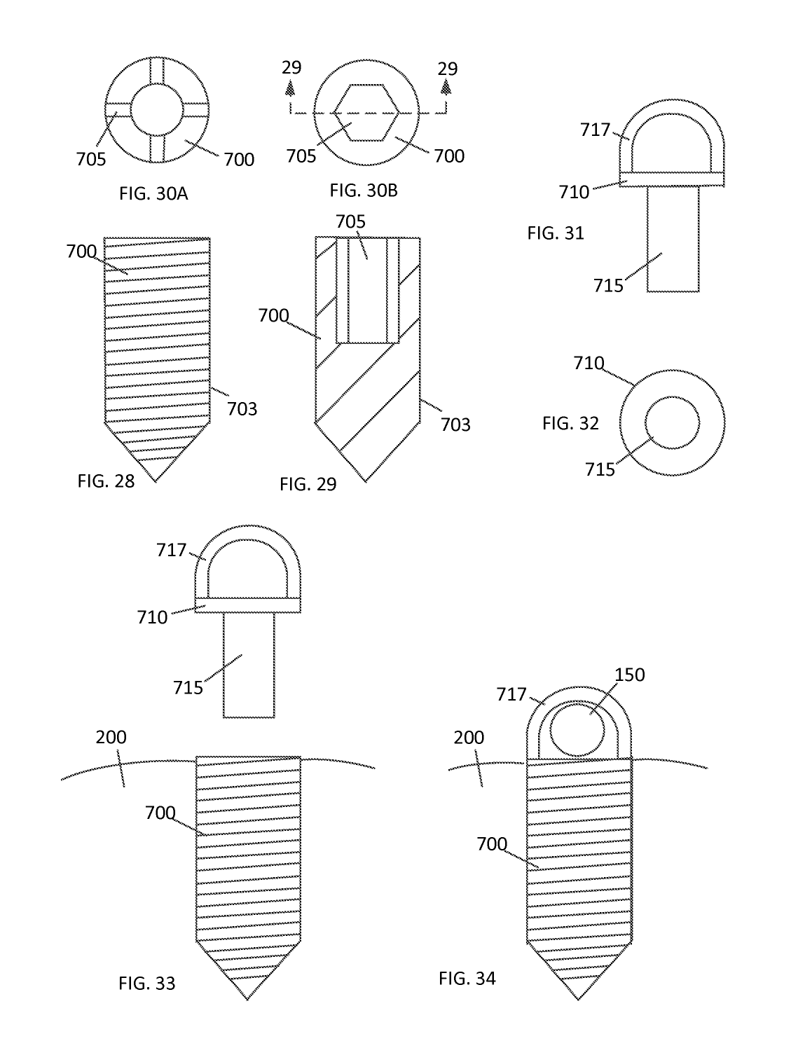

In other embodiments, the cable anchors can be modular structures that include a bone screw portion and a cable anchor portion that is coupled to the top of the bone screw portion. With reference to FIG. 28, a side view of an embodiment of a bone screw 700 is illustrated. The bone screw is elongated and cylindrical in shape with a tapered point. The outer diameter 703 is threaded so that the screw 700 can be screwed into the bone. With reference to FIG. 29, a cross section of the bone screw 700 is illustrated. FIG. 30A a top view of an embodiment the bone screw 700 is illustrated having a recessed cylindrical center and slots for a Phillips head screwdriver as the driving feature 705. FIG. 30B illustrates another embodiment of a top view of the bone screw 700. The driving feature 705 can be formed in the top of the bone screw 700 so that it can be screwed into the bone. In FIG. 30B, the driving feature 705 is a hexagonal recess which can fit around an hex wrench. In other embodiments, the driving feature 705 can be any other type of recessed wrench mechanism such as torx, flat head, flat head, etc.

FIG. 31 illustrates a side view of cable anchor 710 having a cable holding structure 717 and an elongated plug 715 that can fit within the driving feature 705 of the bone screw 700. FIG. 32 illustrates a bottom view of the cable anchor 710. In this embodiment, the elongated plug 715 is a cylindrical structure that can be concentric with the cable anchor 710. In other embodiments, the elongated plug 715 can be any shape that fits closely within the driving feature 705 of the bone screw 700 such as a hexagonal cross section.

With reference to FIGS. 33 and 34, the bone 200 can be drilled and the bone screw 700 can be driven into the bone 200. In this example, the bone screw 700 is driven into the bone 200. The upper edge of the bone screw 700 can be nearly flush with the outer surface of the bone 200. Once the bone screw 700 is inserted into the bone 200, the wrench can be removed from the driving feature and the elongated plug 715 can be inserted into the driving feature 705. A cable 150 can be inserted through the cable holding structure 717. If the elongated plug 715 is cylindrical, the cable anchor 710 will be able to rotated within the bone screw 700.

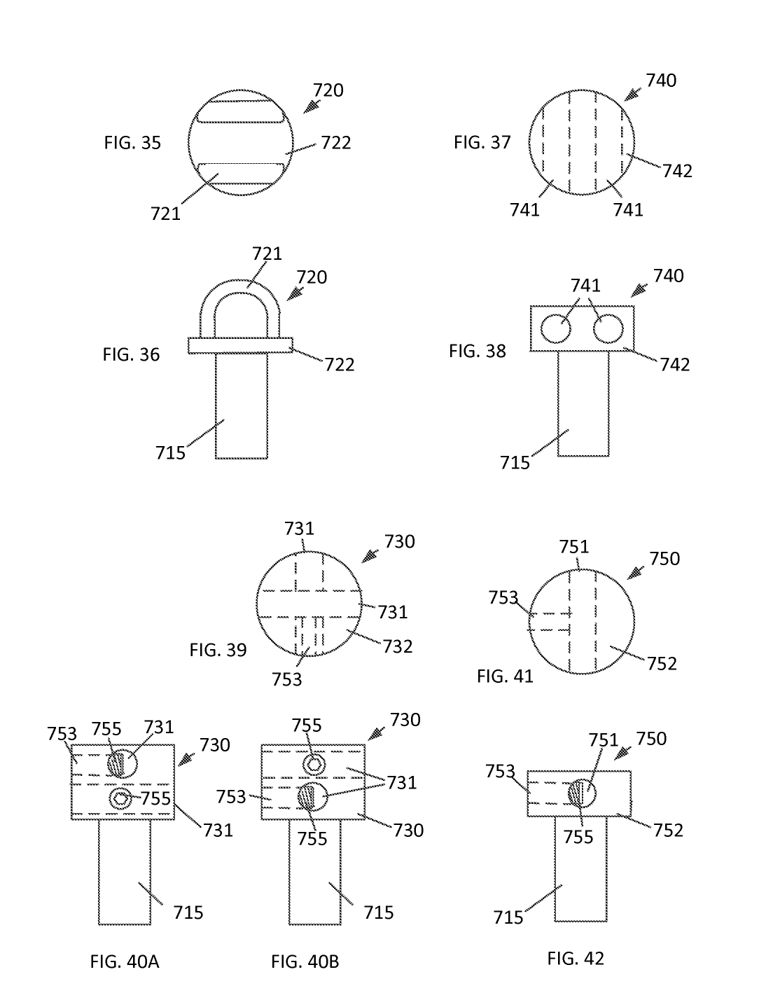

In other embodiments, any other type of cable anchor or cable crimp can be used with the bone screw 700. FIGS. 35-40 illustrate different embodiments of cable anchors that can be inserted into the bone screw 700 as described above. FIG. 35 illustrates a top view of a cable anchor 720 having two cable holders 721 mounted over a circular body 722. The two cable holders 721 can be axially aligned with each other so that one or more cables can pass through both cable holders 721. FIG. 36 illustrates a side view of the cable anchor 720. An elongated plug 715 can be rigidly mounted to the bottom of the body 722. The elongated plug 715 can fit within the driving feature 705 of the bone screw 700.

FIG. 37 illustrates top view of another cable anchor 740 having a body 742 with two parallel cable holes 741. Each of the cable holes 741 can hold a separate cable or a single cable can pass through both cable holes 741. An elongated plug 715 can be rigidly mounted to the bottom of the body 742. FIG. 38 illustrates a side view of the cable anchor 740 showing the parallel cable holes 741 and the elongated plug 715.

FIG. 39 illustrates a top view of another cable anchor 730 having two perpendicular cable holes 731 that cross through the cable anchor body 732. FIGS. 40A and 40B show two side views of the cable anchor 730. The cable holes 731 can be perpendicular but can be on different vertical portions of the cable anchor body 732. The cable holes 731 can be coupled to locking bolts 755 that are within threaded locking holes 753. The locking bolts 755 can be tightened to secure a cable within the cable anchor body 732. An elongated plug 715 can be rigidly mounted to the bottom of the body 732.

FIG. 41 illustrates a top view and FIG. 42 illustrates a side view of an embodiment of a cable crimp 750 with a circular cable crimp body 752. A cable hole 751 extends through the width of the cable crimp body 752. A threaded lock hole 753 can intersect the cable hole 751 and extend through a side of the cable crimp body 752. A locking bolt 755 can be threaded into the lock hole 753 and used to crimp a cable placed through the cable hole 751.

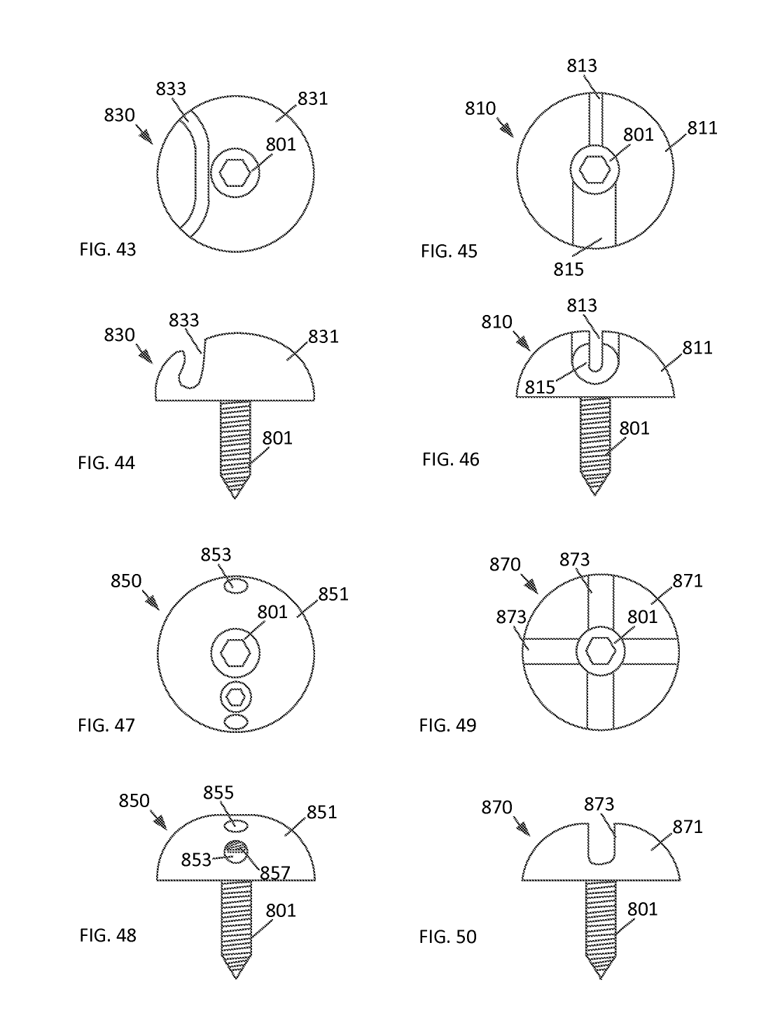

The inventive cable anchors and cable crimps have been described as with the cables passing straight through. This configuration is suitable when the cable is wrapped around the circumference of the bone and the only cable curvature is due to the curvature of the bone. However, in other embodiments inventive cable anchors can be used to alter the direction of the cable. With reference to FIG. 43 a top view of a cable bending anchor 830 is illustrated. In this embodiment, the cable anchor 830 has a circular body 831 and a curved cable slot 833. A bone screw 801 can be placed through a center hole in the cable anchor 830. With reference to FIG. 44 a side view of the cable bending cable anchor 830 is illustrated showing the convex curved upper surface. The curved cable slot 833 can be bend outward towards the bottom of the cable slot 833. This configuration can prevent the cable from being removed from the cable slot 833 when the cable is tensioned. The cable can be dropped into the cable slot 833 rather than being threaded through a cable hole. However in other embodiments, the cable anchor 830 can include a curved cable hole.

FIG. 45 illustrates a top view of a cable crimp 810 having a circular body 811 and a bone screw 801 passing through a center hole in the cable crimp 810. The illustrated cable crimp 810 can be used to hold a cable stop coupled to an end of a cable. The cable slot 813 can be slightly wider than the diameter of the cable and the cable stop slot 815 on the opposite side can be slightly wider than the diameter of the cable stop. FIG. 46 illustrates a side view of a cable crimp 810 and shows the alignment of the cable slot 813 and the cable stop slot 815. The cable crimp 810 can be installed on a bone and the cable stop can be dropped into the cable stop slot 815. When the cable is tensioned, the cable stop will be pressed against the cable slot 813 and the cable will be held stationary by the cable crimp 810.

FIG. 47 illustrates a top view and FIG. 48 illustrates a side view of another embodiment of a cable crimp 850. A cable hole 853 can extend through the cable crimp body 851. A locking hole 855 can intersect the cable hole 853 and a locking screw 857 can be used to hold the cable rigidly within the cable hole 853. The cable crimp body 851 can be circular with a hemispherical surface.

FIG. 49 illustrates a top view and FIG. 50 illustrates a side view of a cable anchor 870 having cable slots 873 that are perpendicular to each other and cross through the body 871 of the cable anchor 870. The cable anchor 870 can be secured to the bone and the cable can be dropped into the slot 873. The cable can be tensioned and the tension can hold the cable within the slot 873.

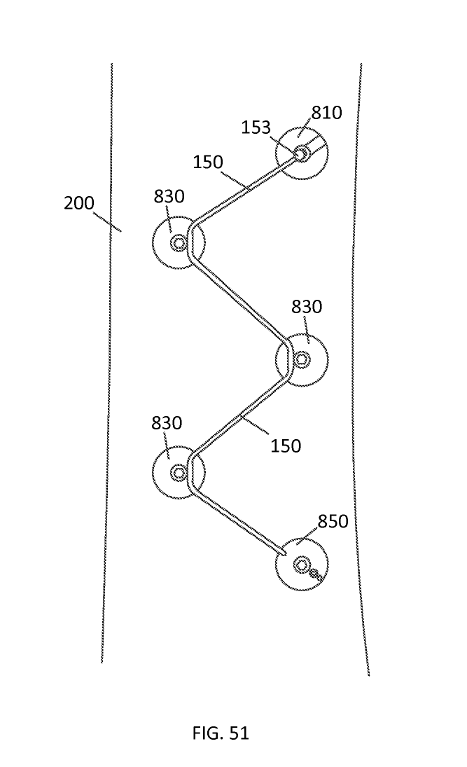

With reference to FIG. 51, the cable bending cable anchors 830 are illustrated in on a bone 200. In this embodiment, the cable bending cable anchors 830 are configured to bend the cable several times across an area of the bone 200. A cable stop 153 can be attached to an end of the cable 150. A cable crimp 810 can hold the cable stop 153 and the cable 150 can extend away from the cable crimp 810. The cable 150 can pass through the slots in the cable anchors 830 and traverse across the width of the bone 200. The cable 150 can pass through a cable crimp 850 and pulled to the required tension. The cable crimp 850 can be used to clamp the cable 150 and maintain the required tension in the cable 150. The cable 150 can then be cut at the exit of the cable crimp 850. In order for the illustrated cable system to be functional, the cable anchors 830 and cable crimps 810, 850 must be rigidly secured to the bone 200 so they will not move when the cable 150 is tensioned. In order to provide the required coupling stength, the cable anchors 830 and cable crimps 810, 850 can be secured to the bone 200 with tacks and/or bone screws or any other suitable bone connecting mechanisms.

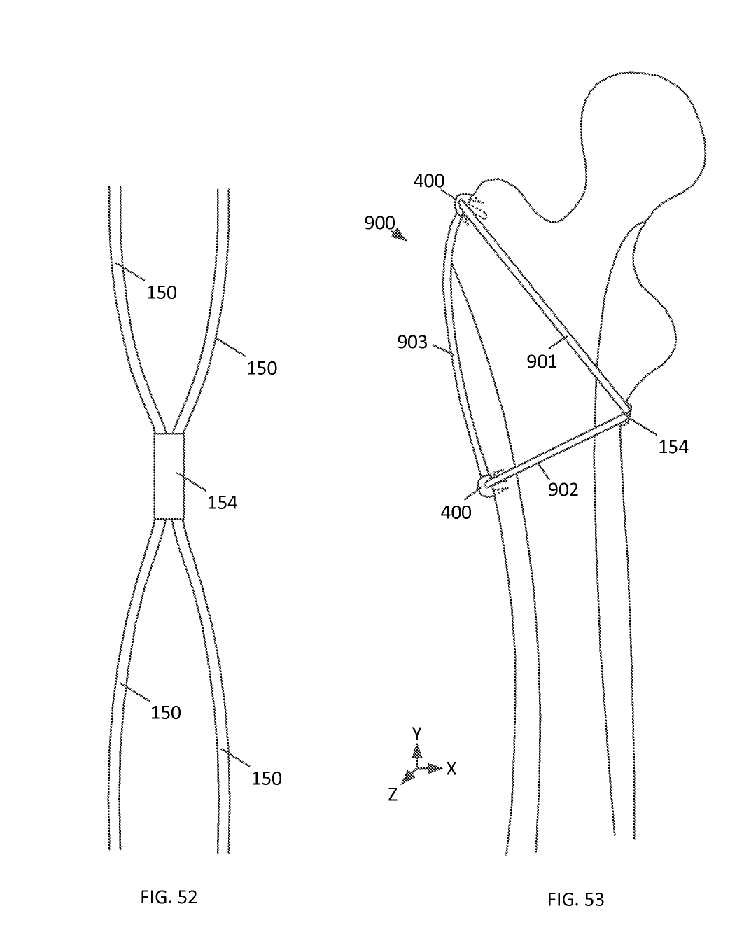

The inventive cable anchor system has been described as using cables that are single tension members. In other embodiments, it is possible to use the inventive anchors with multiple cables that are secured together. With reference to FIG. 52, a pair of cables 150 are illustrated that are rigidly coupled together with a coupler 154 in a center portion of the cables 150. Tension on a single end of the one cable 150 can be transmitted to the other cable through the coupler 154. The coupled cables 150 can be used in various different bone stabilizing applications.

With reference to FIG. 53, a trochenteric cable fixation system 900 is illustrated that can utilize several cables 901, 902, 903 that are coupled to the bone 200 and to each other with cable anchors 400 that can hold multiple cables 901, 902, 903 in tension. The cable anchors 400 can be secured to the bone with tacks and/or bone screws depending upon the most suitable connection mechanism defined by the physician. In FIG. 53, the cables 901, 902 are connected to the coupler 154 which can be adjacent to the lesser trochanter. A first cable 901 can extend around the femur bone 200 from the coupler 154 to an area adjacent to the greater trochanter. Because the cable anchor 400 is rigidly coupled to the surface of the bone 200, it will hold the cable 150 in place and not let it slip which can cause the cable 150 to loosen. The strongest connection between the cable anchor 400 and the bone 200 can be both tacks and bone screws. The cable 901 can also wrap around the opposite of the bone 200 from the connector 154 to the cable anchor 400. A second cable 902 can extend around the bone 200 from the connector 154 to mid portion of the bone 200. The cable anchor 400 can hold the cable 902 in place and prevent movement which can cause the cable 902 to loosen. A third cable 903 can extend between the two cable anchors along a convex surface of the bone 200. The third cable 903 can be tensioned and provide a compressive force under the third cable 150. The cable anchor 400 is shown in more detail in FIG. 14.

The cable system illustrated in FIG. 53 provides compression to the bone 200 because the cables 901, 902, 903 are maintained in tension by the cable anchors 400 that are rigidly coupled to the bone 200. Without the rigid connections between the cable anchors 400 and the bone 200 provided by the tacks and/or bone screws, the cables 901, 902, 903 can easily slip across the bone 200 to a loose position around the bone 200 and the bone compression will be lost. Because the cables 901, 902, 903 can be inelastic, any movement of the cables 901, 902, 903 can result in a complete loss of tension resulting in a non-functional trochenteric cable fixation system 900.

An additional benefit of the inventive cable system is that the circumferential cables 901, 902 are not parallel to each other and are not perpendicular to the center axis of the femur bone 200. This angled cable configuration allows the cables 901, 902 to resist rotational forces applied to the bone 200. For example, if a torsional force was applied to the bone 200 about the center axis or Y axis of the bone 200 with a clockwise force applied to the upper portion of the bone 200 and a counter clockwise rotational force applied to the lower portion of the bone 200, the front portion of the first cable 901 and the back portion of the second cable 902 would be in tension. Because the coupler 154 physically connects these two cables 901, 902, the tension force can be transmitted from the first cable 901 to the second cable 902.

Similarly, bending of the bone 200 can cause the inventive cable system to resist movement. For example, if the upper portion of the femur bone 200 is bent in a counter clockwise rotation about the X-axis, the first cable 901 and the second cable 902 can resist this movement. If the bending force is applied to the upper portion of the bone 200, the portions of the first cable 901 and the second cable 902 behind the bone 200 will be in tension and resist this bending movement. If the upper portion of the femur bone 200 bends in rotation about the Z-axis, the third cable 903 will be tensioned and will resist the bone movement. Without the bone connection of the cable anchors 400, these modified cable configurations would not be possible.

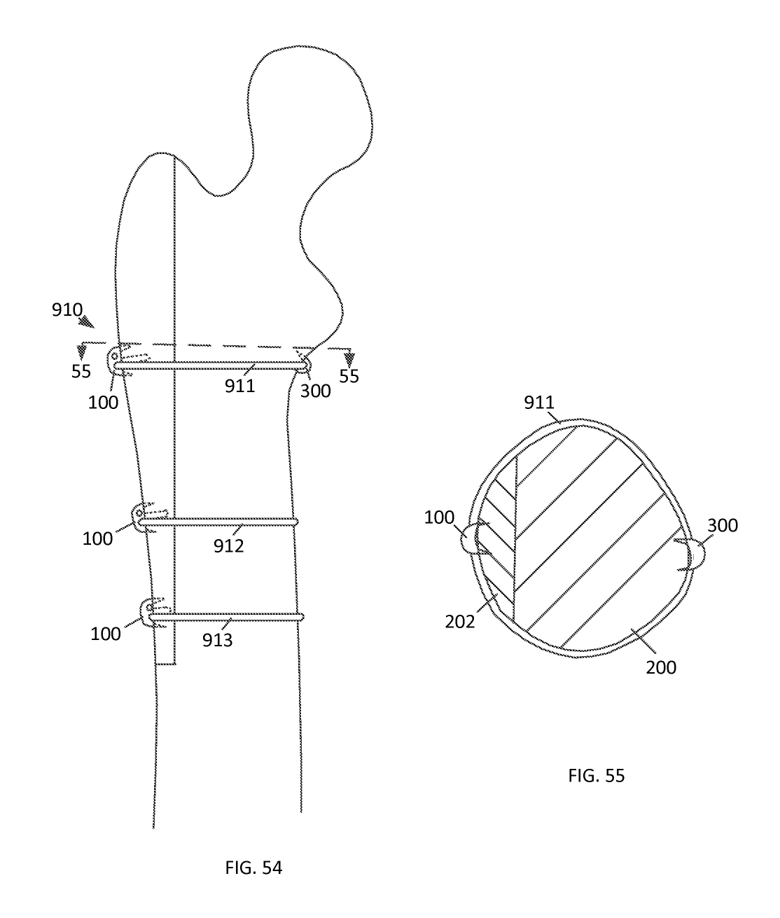

With reference to FIG. 54, a cable system 910 can be used in circumferential configurations. In the illustrated embodiment, the cable system 910 can include three cables 911, 912, 913 that are circumferentially tensioned around a femur bone 200 to secure a structural allograph 202 to the femur bone 200. In this embodiment, a portion of the femur bone 200 can be cut away and the structural allograft 202 can be cut match the cut away portion of the femur bone 200. The structural allograft 202 is then placed against the femur bone 200 at the cut away section and held in place by the cable system 910. The structural allograft 202 must be held in compression against the bone 200 for the injury to heal properly. Each of the cables 911, 912, 913 must be held in tension around the circumference of the bone 200.

The cables 911, 912, 913 can be coupled to cable anchor crimps 100 that are shown in more detail in FIGS. 1-4. The cable anchor crimps 100 can be rigidly attached to the structural allograft 202 with tacks and/or bone screws. Cable stops can be attached to one end of the cables 911, 912, 913 and the cable anchor crimps 100 can be rigidly attached to the cable stops. The cables 911, 912, 913 can then be wrapped around the femur bone 200 and the structural allografts 202 and tensioned to a required force. The opposite end of the cables 911, 912, 913 can be crimped to the cable anchor crimps 100 and the excess cable 911, 912, 913 can be cut away. As discussed, it is critical that the cables 911, 912, 913 not move across the bone. In this example, the lower two cables 912, 913 can be on relatively uniform portions of the bone 200 and structural allograft 202. Thus, the cable anchor crimps 100 coupled to the structural allograft 202 with tacks and/or bone screws can be sufficient to hold the cables 912, 913 in place.

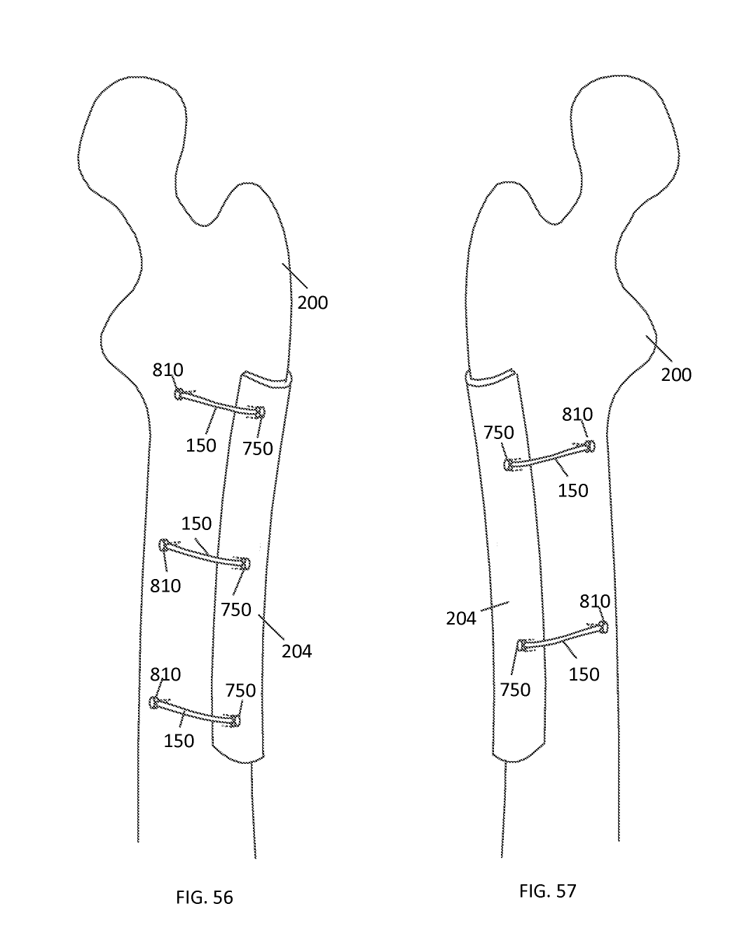

With reference to FIG. 55, the upper cable 911 can be on a tapered section of the femur bone 200 and the structural allografts 202. In order to properly hold the cable 911 in place and prevent slipping, an additional cable anchor 300 may be needed on an opposite side of femur bone 200. Because the cable anchor 300 is rigidly secured to the bone 200 with tacks and/or bone screws, the cable anchor 300 will not slide across the bone 200 to a region having a smaller cross section which would relieve the cable tension. More details of the cable anchor 300 are shown in FIGS. 10-12.

Because the cable anchors and cable crimps can be rigidly coupled to the bones of the patient with tacks and/or bone screws, it may not be necessary for the cable to wrap entirely around the bones. With reference to FIGS. 56 and 57, two sides of a femur bone 200 are illustrated. A strut graft 204 is coupled to the bone 200 and held in place with a plurality of cables 150. The cables 150 can be secured to the femur bone 200 with cable anchors 810 which are illustrated in more detail in FIG. 45. The strut graft 204 can be coupled to several cable crimps 750 shown in more detail in FIG. 42. The cable anchors 810 and cable crimps 750 can be rigidly coupled to the bone 200 with bone screws. In other embodiments, the cable anchors 810 and cable crimps 750 can be rigidly coupled to the bone 200 with both tacks and bone screws.

The cables 150 can be tensioned from the cable anchors 810 and pulled through the cable crimps 750. Once tensioned, the cables 150 can be locked within the cable crimps 750 and the excess cable 150 can be cut away. The inventive cable that does not require the cable to be wrapped around the entire circumference of the bone 200 can have various benefits for the patient. When the cable is wrapped around the back of the bone 200, it can be difficult to know what the cable is being placed against. If the cable is placed against a nerve or blood vessel, this can injure the patient. Since the cable only needs to be coupled to a single side of the bone 200, the physician can more easily avoid nerves, blood vessels and other sensitive body structures. Thus, the illustrated cable system can be particularly useful for areas of the body that have complex bone features.

It will be understood that the inventive system has been described with reference to particular embodiments, however additions, deletions and changes could be made to these embodiments without departing from the scope of the inventive system. Although the systems that have been described include various components, it is well understood that these components and the described configuration can be modified and rearranged in various other configurations.

What is claimed is:

1. A cable anchor comprising:
a cable anchor body having an convex surface that extends over a top and around sides of the cable anchor body and a lower surface that is placed against a bone of a patient;
a center hole that extends through the upper convex surface and the lower surface;
a first cable hole that extends through the cable anchor body from a first side of the upper convex surface to a second side of the upper convex surface;
a bone screw placed through the center hole for securing the cable anchor to the bone of the patient.

2. The cable anchor of claim 1 wherein the center hole is substantially perpendicular to the cable hole.

3. The cable anchor of claim 1 wherein the center hole does not intersect the first cable hole.

4. The cable anchor of claim 1 further comprising:
a second cable hole that extends through the cable anchor body from the first side of the upper convex surface to the second side of the upper convex surface, the second cable hole is substantially parallel to the first cable hole;
wherein the center hole does not intersect the first cable hole or the second cable hole.

5. The cable anchor of claim 4 wherein the second cable hole includes a counter bore section for holding a cable stop coupled to a cable within the second cable hole wherein the cable stop is larger than a diameter of the second cable hole.

6. The cable anchor of claim 1 further comprising:
a locking mechanism for rigidly securing a cable within the first cable hole.

7. The cable anchor of claim 6 wherein the locking mechanism includes a threaded locking hole that extends from the upper convex surface to the first cable hole, wherein the locking hole is substantially perpendicular to the first cable hole.

8. The cable anchor of claim 7 wherein the locking mechanism includes a lock screw having external threads that correspond to the threaded locking hole.

9. The cable anchor of claim 1 wherein the lower surface is concave.

10. The cable anchor of claim 1 further comprising:
a plurality of compression anchor tacks that extend from the lower surface of the cable anchor body and are pressed into the bone of the patient to prevent movement of the cable anchor body.

11. A cable anchor comprising:
a cable anchor body having an upper surface and a lower surface;
a first cable hole that extends through the cable anchor body between the upper surface and the lower surface; and
a plurality of compression anchor tacks that extend from the lower surface of the cable anchor body and are pressed into the bone of the patient to prevent movement of the cable anchor body.

12. The cable anchor of claim 11 further comprising:
a center hole that extends through the upper surface and the lower surface; and
a bone screw placed through the center hole for securing the cable anchor to the bone of the patient;
wherein the center hole is substantially perpendicular to the cable hole.

13. The cable anchor of claim 12 wherein the center hole does not intersect the first cable hole.

14. The cable anchor of claim 11 further comprising:
a second cable hole that extends through the cable anchor body between the upper surface and the lower surface, the second cable hole is substantially parallel to the first cable hole;
wherein the center hole does not intersect the first cable hole or the second cable hole.

15. The cable anchor of claim 14 further comprising:
a third cable hole that extends through the cable anchor body between the upper surface and the lower surface, the third cable hole is substantially parallel to the first cable hole;
wherein the center hole does not intersect the third cable hole.

16. The cable anchor of claim 11 further comprising:
a fourth cable hole that extends through the cable anchor body between the upper surface and the lower surface, the fourth cable hole is substantially perpendicular to the first cable hole;
wherein the center hole does not intersect the first cable hole or the fourth cable hole.

17. A modular cable anchor comprising:
a bone screw having a threaded outer surface, a driving feature for screwing the bone screw into a bone and a recessed hole adjacent to the driving feature;
a cable anchor having a cable holding feature that entends across a cable anchor body and an elongated plug that extends from the cable anchor body;
wherein the elongated plug fits within the recessed hole in the bone screw.

18. The modular cable anchor of claim 17 wherein the cable holding feature is a cable slot.

19. The modular cable anchor of claim 17 wherein the cable holding feature is a cable hole.

20. The cable anchor of claim 19 further comprising:
a cable locking screw within a threaded locking hole that intersects the cable hole.
